(19) United States
(12) Patent Application Publication
(10) Pub. No.: US 2023/0193410 A1
(43) Pub. Date: Jun. 22, 2023

(54) IDENTIFICATION OF NSP1 GENE AS TARGET OF SARS-COV-2 REAL-TIME RT-PCR USING NANOPORE WHOLE GENOME SEQUENCING

(71) Applicant: The University of Hong Kong, Hong Kong (CN)

(72) Inventors: Kelvin Kai-Wang To, Hong Kong (CN); Kwok-Yung Yuen, Hong Kong (CN); Jasper Fuk-Woo Chan, Hong Kong (CN)

(21) Appl. No.: 17/996,287

(22) PCT Filed: Mar. 31, 2021
(86) PCT No.: PCT/CN2021/084357

§ 371 Date: Oct. 14, 2022

Related U.S. Application Data
(60) Provisional application No. 63/013,282, filed on Apr. 21, 2020.

(51) Int. Cl. C12Q 1/70

(52) U.S. Cl. CPC C12Q 1/701; C12Q 2600/16

(57) ABSTRACT

Sequences for detection of SARS-CoV-2, probes and primers that target the target sequences, and methods of use thereof for the detection and diagnosis of SARS-CoV-2 are provided. Detection methods include, but are not limited to, microarray, differential display, RNase protection assay, northern blot, reverse transcriptase (RT) polymerase chain reaction (PCR), and combinations thereof. In preferred embodiments, the detection methods include RT-PCT, more preferably realtime or quantitative RT-PCR, most preferably wherein the RT-PCR includes target specific reverse transcription and/or target specific PCR. In some embodiments, the disclosed primers, probes, compositions, or methods are more sensitive, selective, or combination thereof for SARS-CoV-2 relative to one or more other human- and/or non-human pathogenic coronaviruses and/or respiratory pathogens.

### FIELD OF THE INVENTION

The present invention is generally in the field of detecting SARS-CoV-2.

### BACKGROUND OF THE INVENTION

In 2003, severe acute respiratory syndrome coronavirus (SARS-CoV) cross species barrier and became the first coronavirus to cause high fatality rate in humans [1]. Most patients with coronavirus disease 2019 (COVID-19) present with respiratory symptoms, while 18% exhibit gastrointestinal symptoms [4]. Radiologically, COVID-19 is characterized by multifocal and 15 peripheral ground glass opacities, but 2.9% of severe cases did not show any abnormalities in their lung computed tomography [2,5]. Complications include acute respiratory distress syndrome, arrhythmia, secondary bacterial infection, and multiorgan failure [6-9]. Pathologically, COVID-19 was characterized by diffuse alveolar damage, interstitial lymphocyte infiltrates, and multinucleated 20 syncytial cells in the lung, and microvascular steatosis in the liver [10].

The importance of diagnostic testing is well demonstrated with COVID-19. Due to a shortage of diagnostic tests, a large number of undiagnosed patients with relatively mild symptoms were unknowingly spreading the virus in the community, and has led to the large outbreaks in some countries [11]. 25 Undocumented infection has been estimated to be the source of 79% of laboratory-confirmed infections [12].

Laboratory confirmation of SARS-CoV-2 relies on accurate molecular assays. Many groups have shared their in-house real-time reverse transcription-polymerase chain reaction (RT-PCR) protocol with the World Health 30 Organization during the early period of COVID-19, which has tremendously helped clinical microbiology laboratories around the world in the detection of SARS-CoV-2 [13]. Currently, the gene targets of real-time reverse transcription polymerase chain reaction (RT-PCR) for SARS-CoV-2 include the open reading frame 1a or 1b (ORF1a or 1b), RNA-dependent RNA polymerase (RdRp)/helicase (Hel), spike (S), envelope (E), and nucleocapsid (N) genes [13,14]. The choice of these gene targets were based on previous experience with SARS-CoV and Middle East respiratory syndrome coronavirus (MERS-CoV) [15,16].

Similar to other RNA respiratory viruses, SARS-CoV-2 can mutate quickly due to the intrinsic infidelity of viral RNA polymerase. The mean evolutionary rate of SARS-CoV-2 has been estimated to be 1.8×10⁻³ substitutions per site per year [17]. Mutations arising at the current gene targets can lower the sensitivity of the existing assays. Currently, 3 major variants have been identified, including 2 subclusters of variant A (ancestral type, characterized by T29095C of N gene), variant B (characterized by T8782C of nsp4 gene and C28144T of orf8 gene) and variant C (characterized by G26144T of orf3a gene) [18]. According to an analysis by GISAID (https://www.epicov.org/epi3/frontend#lightbox1588038909), mutations have been found in binding sites of primers and probes of published RT-PCR protocols. The percent of genomes with mutation in the primer region was as high as 18%. Therefore, there is an urgent need to expand the number of gene targets that can be used for RT-PCR diagnosis.

It is an object of the present invention to provide compositions, methods, and kits for detecting and diagnosing SARS-CoV-2.

It is a further object of the present invention to provide compositions, methods, and kits for detecting and diagnosing SARS-CoV-2 which show improved sensitivity and specificity relative to existing detection methods.

### SUMMARY OF THE INVENTION

Sequences for detection of SARS-CoV-2 are provided and can, for example, include or consist of a sequence of any of CATTCAGTACGGTCGTAGTGGTGAG (SEQ ID NO: 1), CCTTGCGGTAAGCCACTGGTA (SEQ ID NO:2), CCCACATGAGGGACAAGGACACCA (SEQ ID NO:3), a sequence having at least 70%, 75%, 80%, 85%, 90%, 95%, 96%, 97%, 98%, or 99% sequence identity thereto, a nucleic acid sequence having 1, 2, 3, 4, 5, 6, 7, 8, 9, or 10 nucleic acid substitution(s), addition(s), deletion(s), or a combination thereof relative thereto, or the reverse complement of any of the foregoing.

The sequences can be targeted by probes and primers. Thus, probes and primers that target the foregoing target sequences and methods of use thereof for the detection and diagnosis of SARS-CoV-2 are also provided. In some embodiments, the primers or probes hybridizes with a sequence of any of SEQ ID NOS: 1-3, a sequence having at least 70%, 75%, 80%, 85%, 90%, 95%, 96%, 97%, 98%, or 99% sequence identity thereto, a nucleic acid sequence having 1, 2, 3, 4, 5, 6, 7, 8, 9, or 10 nucleic acid substitution(s), addition(s), deletion(s), or a combination thereof relative thereto, or the reverse complement of any of the foregoing.

Particularly preferred primer sets and probes are as SEQ ID Nos: 1-3 and can be used alone or in combination. In some embodiments, the probes are modified to include a detectable reporter such as a radioactive or fluorescent label. In particularly embodiments, the probe are modified for use a realtime polymerase chain reaction, and include, for example, one or more fluorescent reporters, one or more quenchers, or a combination thereof. In a non-limiting example, the probes exemplified in Table 1 include a 5′ fluorescent reporter and a 3′ quencher.

The target sequences, primers, and probes can use in methods of detecting SARS-CoV-2 nucleic acids in a sample such as mucus, sputum (processed or unprocessed), bronchial alveolar lavage (BAL), bronchial wash (BW), bodily fluids, cerebrospinal fluid (CSF), urine, tissue (e.g., biopsy material), rectal swab, nasopharyngeal aspirate, nasopharyngeal swab, throat swab, feces, plasma, serum, or whole blood, thus, methods of detecting SARS-CoV-2 in such samples are also provided. The sample can be one that is isolated from a subject that may have been exposed to or is suspected of having SARS-CoV-2. In some embodiments, the sample is processed to expose or isolate nucleic acids from sample before it is subjected to the detection method.

Detection methods include, but are not limited to, microarray, differential display, RNase protection assay, northern blot, reverse transcriptase (RT) polymerase chain reaction (PCR), and combinations thereof. In preferred embodiments, the detection methods include RT-PCT, more preferably realtime or quantitative RT-PCR, most preferably wherein the RT-PCR includes target specific reverse transcription and/or target specific PCR. In preferred embodiments, the methods can be used to detect one or more of the sequences for detection disclosed herein, including, but not limited to any one of SEQ ID NOs:1-3, and the reverse complements thereof. Preferred primer sets for reverse transcription and/or PCR include: SEQ ID NOS: 1 and 2, which can optionally be used in combination with a probe of SEQ ID NO:3. Any of the foregoing sets or primers and optionally probes can be used in combinations of 2 or even 3 for multiplex reactions. Detection can include identification of one or more amplicons formed by PCR utilizing one or more of the primer pairs, optionally via detection of fluorescence from the probe.

In some embodiments, the assays are multiplexed to detect two or more target sequences (e.g., RdRp/helicase (Hel), Spike (S), E, ORF1a/b and/or Nucleocapsid (N)), in addition to NSP1, at once. SARS-CoV-2 has four structural proteins, known as the S (spike), E (envelope), M (membrane), and N (nucleocapsid) proteins; the N protein holds the RNA genome, and the S, E, and M proteins together create the viral envelope. The incorporation of the NSP1 gene in multiplex RT-PCR assays can be used to improve the detection of SARS-CoV-2 using these other targets.

In some embodiments, the disclosed primers, probes, compositions, or methods are more sensitive, selective, or combination thereof for SARS-CoV-2 relative to one or more other human- and/or non-human pathogenic coronaviruses and/or respiratory pathogens, such as SARS-CoV, MERS-CoV, HCoV-229E, HCoV-NL63, HCoV-OC43), and 12 virus culture isolates of other respiratory viruses (Influenza virus A[H1N1] and A[H3N2], influenza B virus, influenza C virus, rhinovirus, adenovirus, respiratory syncytial virus, human metapneumovirus and parainfluenza virus types 1-4. Thus, in some embodiments, one or more non-SARS-CoV-2 virus cannot be detected according the disclosed compositions or methods. In particular embodiments, the undetectable non-SARS-CoV-2 virus is SARS-CoV.

Methods of diagnosing a subject with SARS-CoV-2 are also provided and can include analyzing a sample from the subject according to a detection method, wherein detection of SARS-CoV-2 in the sample indicates the subject has a SARS-CoV-2 infection.

### BRIEF DESCRIPTION OF THE DRAWINGS

FIG. 1 is an alignment of our novel nsp1 primers and probes with SARS-CoV-2 and other human coronaviruses in the genus Betacoronavirus.

FIG. 2 shows coverage map of the nanopore sequencing of SISPA-amplified viral genome. X-axis shows the nucleotide position, while Y axis shows the number of reads. The coverage map was generated by integrative genomics viewer.

FIG. 3 is a bar graph showing coverage information of nanopore sequencing for each real-time RT-PCR target region. The mean coverage of each RT-PCR amplicon is expressed as the % of the mean coverage of the entire N gene (nucleotide position 28274 to 29533). Error bar indicates one standard deviation.

### DETAILED DESCRIPTION OF THE INVENTION

### I. Definitions

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. Thus, if a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. Further, each of the materials, compositions, components, etc. contemplated and disclosed as above can also be specifically and independently included or excluded from any group, subgroup, list, set, etc. of such materials. These concepts apply to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

The terms “complement”, “complementary” or “complementarity” as used herein with reference to polynucleotides (i.e., a sequence of nucleotides such as an oligonucleotide or a target nucleic acid) refer to the Watson/Crick base-pairing rules. The complement of a nucleic acid sequence as used herein refers to an oligonucleotide which, when aligned with the nucleic acid sequence such that the 5′ end of one sequence is paired with the 3′ end of the other, is in “antiparallel association.” For example, the sequence “5′-A-G-T-3” is complementary to the sequence “3‘-T-C-A-S’.” Certain bases not commonly found in naturally-occurring nucleic acids may be included in the nucleic acids described herein. These include, for example, inosine, 7-deazaguanine, Locked Nucleic Acids (LNA), and Peptide Nucleic Acids (PNA). Complementarity need not be perfect; stable duplexes may contain mismatched base pairs, degenerative, or unmatched bases. Those skilled in the art of nucleic acid technology can determine duplex stability empirically considering a number of variables including, for example, the length of the oligonucleotide, base composition and sequence of the oligonucleotide, ionic strength and incidence of mismatched base pairs. A complement sequence can also be an RNA sequence complementary to the DNA sequence or its complement sequence, and can also be a cDNA.

The term “substantially complementary” as used herein means that two sequences hybridize. In some embodiments, the hybridization occurs only under stringent hybridization conditions. The skilled artisan will understand that substantially complementary sequences need not hybridize along their entire length. In particular, substantially complementary sequences may comprise a contiguous sequence of bases that do not hybridize to a target sequence, positioned 3′ or 5′ to a contiguous sequence of bases that hybridize under stringent hybridization conditions to a target sequence.

The term “hybridize” as used herein refers to a process where two substantially complementary nucleic acid strands (at least about 65% complementary over a stretch of at least 14 to 25 nucleotides, at least about 75%, or at least about 90% complementary) anneal to each other under appropriately stringent conditions to form a duplex or heteroduplex through formation of hydrogen bonds between complementary base pairs. Hybridizations are typically and preferably conducted with probe-length nucleic acid molecules, preferably 15-100 nucleotides in length, more preferably 18-50 nucleotides in length. Nucleic acid hybridization techniques are well known in the art. See, e.g., Sambrook, et al., 1989, Molecular Cloning: A Laboratory Manual, Second Edition, Cold Spring Harbor Press, Plainview, N.Y. Hybridization and the strength of hybridization (i.e., the strength of the association between the nucleic acids) is influenced by such factors as the degree of complementarity between the nucleic acids, stringency of the conditions involved, and the thermal melting point (T.sub.m) of the formed hybrid. Those skilled in the art understand how to estimate and adjust the stringency of hybridization conditions such that sequences having at least a desired level of complementarity will stably hybridize, while those having lower complementarity will not. For examples of hybridization conditions and parameters, see, e.g., Sambrook, et al., 1989, Molecular Cloning: A Laboratory Manual, Second Edition, Cold Spring Harbor Press, Plainview, N.Y.; Ausubel, F. M. et al. 1994, Current Protocols in Molecular Biology, John Wiley & Sons, Secaucus, N.J. In some embodiments, specific hybridization occurs under stringent hybridization conditions. An oligonucleotide or polynucleotide (e.g., a probe or a primer) that is specific for a target nucleic acid will “hybridize” to the target nucleic acid under suitable conditions.

As used herein, the term “primer” refers to an oligonucleotide, which is capable of acting as a point of initiation of nucleic acid sequence synthesis when placed under conditions in which synthesis of a primer extension product which is complementary to a target nucleic acid strand is induced, i.e., in the presence of different nucleotide triphosphates and a polymerase in an appropriate buffer (“buffer” includes pH, ionic strength, cofactors etc.) and at a suitable temperature. One or more of the nucleotides of the primer can be modified for instance by addition of a methyl group, a biotin or digoxigenin moiety, a fluorescent tag or by using radioactive nucleotides. A primer sequence need not reflect the exact sequence of the template. For example, a non-complementary nucleotide fragment may be attached to the 5′ end of the primer, with the remainder of the primer sequence being substantially complementary to the strand. The term primer as used herein includes all forms of primers that may be synthesized including peptide nucleic acid primers, locked nucleic acid primers, phosphorothioate modified primers, labeled primers, and the like. The term “forward primer” as used herein means a primer that anneals to the anti-sense strand of double-stranded DNA (dsDNA). A “reverse primer” anneals to the sense-strand of dsDNA.

Primers are typically at least 10, 15, 18, or 30 nucleotides in length or up to about 100, 110, 125, or 200 nucleotides in length. In some embodiments, primers are preferably between about 15 to about 60 nucleotides in length, and most preferably between about 25 to about 40 nucleotides in length. In some embodiments, primers are 15 to 35 nucleotides in length. There is no standard length for optimal hybridization or polymerase chain reaction amplification. An optimal length for a particular primer application may be readily determined in the manner described in H. Erlich, PCR Technology, PRINCIPLES AND APPLICATION FOR DNA AMPLIFICATION, (1989).

As used herein, the term “primer pair” refers to a forward and reverse primer pair (i.e., a left and right primer pair) that can be used together to amplify a given region of a nucleic acid of interest.

“Probe” as used herein refers to a nucleic acid that interacts with a target nucleic acid via hybridization. A probe may be fully complementary to a target nucleic acid sequence or partially complementary. The level of complementarity will depend on many factors based, in general, on the function of the probe. Probes can be labeled or unlabeled, or modified in any of a number of ways well known in the art. A probe may specifically hybridize to a target nucleic acid. Probes may be DNA, RNA or a RNA/DNA hybrid. Probes may be oligonucleotides, artificial chromosomes, fragmented artificial chromosome, genomic nucleic acid, fragmented genomic nucleic acid, RNA, recombinant nucleic acid, fragmented recombinant nucleic acid, peptide nucleic acid (PNA), locked nucleic acid, oligomer of cyclic heterocycles, or conjugates of nucleic acid. Probes may comprise modified nucleobases, modified sugar moieties, and modified internucleotide linkages. Probes are typically at least about 10, 15, 20, 25, 30, 35, 40, 50, 60, 75, 100 nucleotides or more in length.

As used herein, the term “sample” refers to in vitro as well as clinical samples obtained from a patient. In preferred embodiments, a sample is obtained from a biological source (i.e., a “biological sample”), such as tissue or bodily fluid collected from a subject. Sample sources include, but are not limited to, mucus, sputum (processed or unprocessed), bronchial alveolar lavage (BAL), bronchial wash (BW), blood, bodily fluids, cerebrospinal fluid (CSF), urine, plasma, serum, or tissue (e.g., biopsy material), nasopharyngeal aspirate, nasopharyngeal swab, throat swab, and other discussed herein and otherwise known in the art.

The term “specific” as used herein in reference to an oligonucleotide primer means that the nucleotide sequence of the primer has at least 12 bases of sequence identity with a portion of the nucleic acid to be amplified when the oligonucleotide and the nucleic acid are aligned. An oligonucleotide primer that is specific for a nucleic acid is one that, under the stringent hybridization or washing conditions, is capable of hybridizing to the target of interest and not substantially hybridizing to nucleic acids which are not of interest. Higher levels of sequence identity are preferred and include at least 75%, at least 80%, at least 85%, at least 90%, at least 85-95%, and more preferably at least 98% sequence identity. Sequence identity can be determined using a commercially available computer program with a default setting that employs algorithms well known in the art. As used herein, sequences that have “high sequence identity” have identical nucleotides at least at about 50% of aligned nucleotide positions, preferably at least at about 60% of aligned nucleotide positions, and more preferably at least at about 75% of aligned nucleotide positions.

“Sensitivity” as used herein, is a measure of ability of a detection assay to directly or indirectly detect the presence of a target sequence (e.g., a SARS-CoV-2 viral sequence) in a sample.

“Specificity,” as used herein, is a measure of the ability of a detection assay to distinguish a truly occurring target sequence (e.g., a SARS-CoV-2 viral sequence) from other closely related sequences (e.g., other human-pathogenic coronaviruses and respiratory pathogens). It is the ability to avoid false positive detections.

The term “stringent hybridization conditions” as used herein refers to hybridization conditions at least as stringent as the following: hybridization in 50% formamide, 5×SSC, 50 mM NaH2PO4, pH 6.8, 0.5% SDS, 0.1 mg/mL sonicated salmon sperm DNA, and 5× Denhart's solution at 42 C. overnight; washing with 2×SSC, 0.1% SDS at 45 C.; and washing with 0.2×SSC, 0.1% SDS at 45 C. In another example, stringent hybridization conditions should not allow for hybridization of two nucleic acids which differ over a stretch of 20 contiguous nucleotides by more than two bases.

The terms “target nucleic acid” or “target sequence” or “target segment” as used herein refer to a nucleic acid sequence of interest to be detected and/or quantified in the sample to be analyzed. Target nucleic acid may be composed of segments of a genome, a complete gene with or without intergenic sequence, segments or portions of a gene with or without intergenic sequence, or sequence of nucleic acids to which probes or primers are designed to hybridize. Target nucleic acids may include a wild-type sequence(s), a mutation, deletion, insertion or duplication, tandem repeat elements, a gene of interest, a region of a gene of interest or any upstream or downstream region thereof. Target nucleic acids may represent alternative sequences or alleles of a particular gene. Target nucleic acids may be derived from genomic DNA, cDNA, or RNA.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each claim of this application.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

### II. Compositions

A. Target, Probe, and Primer Sequences

Primers and probes for use in the detection of the gene encoding NSP1 of SARS-CoV-2 are provided. Although sometime referred to herein is a probe or a primer, it will be appreciated that any of the probe sequences and the reverse complements thereof can be used as primer sequences, and any of the primer sequences and the reverse complements thereof can be used as probe sequences. All of the probes are expressly provided with and without detection labels (e.g., fluorophores, etc.)

The positive-sense, single-stranded RNA genome of SARS-CoV-2 is ˜30 kilobases in size and encodes ˜9860 amino acids. The disclosed probes and primers are typically designed to hybridize with a target SARS-CoV-2 genomic sequence, or the reverse complement thereof that can be generated by reverse transcription. In some embodiments, the SARS-CoV-2 genomic sequence is the sequence of GenBank accession no. MN975262. In some embodiments, the SARS-CoV-2 genomic sequence is the sequence of GenBank accession no. MN908947.3. The disclosed primers and probes are used to detect the 3 major SARS-CoV-2 variants, including variant A (ancestral type), B (characterized by T8782C of nsp4 gene and C28144T of orf8 gene) and C (characterized by T29095C of N gene).

The target nucleic acid to which the probes and/or primer hybridize can be single stranded or double stranded RNA or DNA (e.g., single stranded or duplex cDNA), and can be genomic (positive strand) sequence, or the reverse complement thereof. In some embodiments, the probe(s) and/or primer(s) are can detect and/or facilitate transcription, strand synthesis, and/or amplification of the target gene (NSP1) from any SARS-CoV-2, variant.

In some embodiments, the probes and primers do not hybridize, do not hybridize under stringent conditions, are unsuitable for detection, transcription, strand synthesis, and/or amplification of the corresponding or homologous target genomic sequence, or any combination thereof, of one or more other human- and/or non-human pathogenic coronaviruses or respiratory pathogens, including, but not limited to, SARS-CoV, MERS-CoV, HCoV-OC43, HCoV-229E, HCoV-NL63, adenovirus, human metapneumovirus, influenza A (H1N1 and H3N2) viruses, influenza B virus, influenza C virus, influenza viruses (A[H1N1], A[H3N2], B, C), respiratory syncytial virus, parainfluenza viruses 1-4, human metapneumovirus, rhinovirus/enterovirus and adenovirus.

For the sequences herein, N denotes A, G, T/U, or C; R denotes A or G; Y denotes T/U or C; K denotes G or T/U; and W denotes A or T/U. For all DNA sequences herein, the corresponding RNA sequence is also expressly provided. For all nucleic acid sequences provided herein, the corresponding complementary sequence and reverse complementary sequence are also expressly provided.

B. Modifications to Probes and Primers

Any of the probes and primers can include one or more modifications to enhance, improve or facilitate its desired function. Common modifications include those that enhance detection including radioactive labels (radiolabels), fluorescent reporters (e.g., fluorophores and/or quenchers), attachment moieties (e.g., amine, glycerol, phosphate, thiol, etc.), binding moieties (e.g., biotin, digoxigenin, dinitrophenol, etc.), and/or antisense enhancers, or are spacers, analogs, intercalation agents, or phosphorothioates. Modifications can be used in any way suitable for performing the desired function. For example, modifications can be made at the 3′ end, the 5′ end, internally, or any combination thereof of the primer or probe.

Fluorophore and quencher modifications, particularly those suitable for detection during realtime PCR, are particularly advantageous for the disclosed compositions, particularly probes. Single-quenched and double-quenched probes are contemplated. Double-quenched probes may provide consistently lower background, resulting in higher signal compared to single-quenched probes. Double-quenched probes may include, e.g., ZEN™ or TAO™ molecules as a secondary, internal quencher allowing for longer probe lengths to be used in addition to providing strong quenching and increased signal.

Exemplary fluorescent modifications include, but are not limited to, 6-FAM™ (fluorescein), ROX™, Cyanine 3, Cyanine 5, Cyanine 5.5, 6-FAM dT, HEX™, JOE™, 6-Carboxy-rhodamine 6G™, TAMRA, TAMRA NHS Ester, TET™, TxRd, (Sulforhodamine 101-X), A488 (Sulfonated Fluorescein 488), WellRED D2-PA, WellRED D3-PA, and WellRED D4-PA.

Traditional dark quenchers that absorb broadly and do not emit light, which allows use of multiple reporter dyes with the same quencher. This characteristic allows for expanded options for multiplex assays. Dark quenchers reduce signal cross-talk, simplifying reporter dye detection, making them compatible with a broad range of image analysis instruments. Examples of dark quenchers include Black Hole Quenchers, and Iowa Black FQ and RQ, and the internal ZEN Quencher. Other suitable quenchers include, but are not limited to, BHQ1 and IABkFQ.

### III. Methods of Use

The disclosed target sequences, probes, and primers can be used in methods of detecting SARS-CoV-2. Methods typically involve directly or indirectly detecting virus (e.g., viral genome) in a biological sample.

A. Samples and Sample Preparation

Biological samples include, but are not limited to, tissue or bodily fluid collected from a subject having or suspected of having the virus. Sample sources include, but are not limited to, mucus, sputum (processed or unprocessed), bronchial alveolar lavage (BAL), bronchial wash (BW), blood, bodily fluids, cerebrospinal fluid (CSF), urine, plasma, serum, or tissue (e.g., biopsy material). Preferred sample sources include mucus, rectal swab, nasopharyngeal aspirate, nasopharyngeal swab, throat swab, feces, sputum, plasma, serum, or whole blood. In some embodiments, the sample is treated with a DNAase.

The methods may include sample preparation. Sample preparation methods for detection of virus in biological samples are known in the art, and exemplified below. For example, in some embodiments, total nucleic acid (TNA) extraction of clinical specimens and laboratory cell culture of viral isolates are performed using a kit such as NucliSENS easyMAG extraction system. The volume of the specimens used for extraction and the elution volume depended on the specimen type and the available amount of the specimen.

B. Methods of Detection

Methods of using the disclosed primers and probes to detect virus in a sample are known and include, for example, microarrays, differential display, RNase protection assays, northern blot, and RT-PCR.

In preferred embodiments, the method of detection is reverse transcriptase (RT) polymerase chain reaction (PCR), preferably target sequence-specific RT-PCR, most preferably, target sequence-specific quantitative or realtime RT-PCR. RT-PCR is a variant of polymerase chain reaction (PCR), a laboratory technique commonly used in molecular biology to generate many copies of a deoxyribonucleic acid (DNA) sequence, a process termed “amplification.” In RT-PCR, a ribonucleic acid (RNA) strand is first reverse transcribed into its DNA complement (complementary DNA, or cDNA) using the enzyme reverse transcriptase. The resulting cDNA is subsequently amplified using traditional PCR. RT-PCR utilizes a pair of primers, which are complementary to a defined sequence on each of the two strands of the cDNA. These primers are then extended by a DNA polymerase and a copy of the strand is made after each PCR cycle, leading to exponential amplification. It has been discovered that the disclosed compositions, methods and kits provide alternate gene regions of the SARS-CoV-2 to target in an RT-PCR assay, as well as design of probes used in RT-PCR, that result in an RT-PCR method for detecting SARS-CoV-2 that has improved sensitivity and specificity over alternative methods.

A reverse transcription (RT) reaction refers to the process in which single-stranded RNA is reverse transcribed into complementary DNA (cDNA) by using total cellular RNA or poly(A) RNA, a reverse transcriptase enzyme, one or more primers, dNTPs (refers to a mixture of equal molar of dATP, dTTP, dCTP, and dGTP), and typically an RNase inhibitor. Primers for an RT reaction can be random primers (e.g., random hexamers) or oligo dT for cDNA production from total RNA or polyA RNA (mRNA) respectively, or can be sequence-specific to drive selective cDNA preparation of only a target sequence or sequence(s). The disclosed methods typically include sequence specific RT primer(s). Exemplary primers those disclosed above.

General methods and kits including reaction components for reverse transcription are known and the art and can be employed in the disclosed methods.

A typical reaction mixture includes RNA, primer, dNTP nucleotide mixture, reverse transcriptase, RNase inhibitor, buffer including Tris-HCl, KCl, MgCl₂, DTT, and nuclease free water up to the desired reaction volume.

Next, PCR can be used for second strand synthesis (e.g., to form double stranded cDNA amplicons), and for amplification of the cDNA template. PCR typically relies on a forward and reverse primer (e.g., a primer set). Preferably, the forward and reverse primers specifically amplify the target region whose detection or quantification is desired. In some embodiments, at least one of the PCR primers is the same as at least one of the RT primers.

Other reagents for second strand synthesis and PCR can include, but are not limited to, a DNA polymerase (e.g., heat-resistant Taq polymerase), dNTPs, a buffer solution providing a suitable chemical environment for optimum activity and stability of the DNA polymerase, bivalent cations, typically magnesium (Mg) or manganese (Mn) ions, etc.

The RT reaction and PCR cycle(s) can be carried out as separate and distinct reactions, or in a single tube using a thermocycler as is known in the art and exemplified below.

In preferred embodiments, the RT-PCR is quantitative or realtime PCR. Such assays include non-specific detection: real-time PCR with double-stranded DNA-binding dyes as reporters, where a DNA-binding dye binds to all double-stranded (ds) DNA in PCR, increasing the fluorescence quantum yield of the dye. An increase in DNA product during PCR therefore leads to an increase in fluorescence intensity measured at each cycle. However, dsDNA dyes such as SYBR Green will bind to all dsDNA PCR products, including nonspecific PCR products (such as Primer dimer). This can potentially interfere with, or prevent, accurate monitoring of the intended target sequence.

In real-time PCR with dsDNA dyes the reaction is prepared as usual, with the addition of fluorescent dsDNA dye. Then the reaction is run in a real-time PCR instrument, and after each cycle, the intensity of fluorescence is measured with a detector; the dye only fluoresces when bound to the dsDNA (i.e., the PCR product). This method has the advantage of only needing a pair of primers to carry out the amplification, which keeps costs down; multiple target sequences can be monitored in a tube by using different types of dyes.

In preferred embodiments, the detect assay is specific detection by realtime RT-PCR carried out using a fluorescent reporter probe. Florescent reporter probes detect only the DNA containing the sequence complementary to the probe; therefore, use of the reporter probe significantly increases specificity, and enables performing the technique even in the presence of other dsDNA. Using different-colored labels, fluorescent probes can be used in multiplex assays for monitoring several target sequences in the same tube. The specificity of fluorescent reporter probes also prevents interference of measurements caused by primer dimers, which are undesirable potential by-products in PCR.

The method relies on a DNA-based probe with a fluorescent reporter at one end and a quencher of fluorescence at the opposite end of the probe. Suitable probe sequences, and well as exemplary fluorescent reports and quenchers are discussed above.

The close proximity of the reporter to the quencher prevents detection of its fluorescence; breakdown of the probe by the 5′ to 3′ exonuclease activity of the (e.g., Taq) polymerase breaks the reporter-quencher proximity and thus allows unquenched emission of fluorescence, which can be detected after excitation with a laser. An increase in the product targeted by the reporter probe at each PCR cycle therefore causes a proportional increase in fluorescence due to the breakdown of the probe and release of the reporter.

The RT-PCR is prepared as is known in the art and exemplified below, and the reporter probe is added. As the reaction commences, during the annealing stage of the PCR both probe and primers anneal to the DNA target. Polymerization of a new DNA strand is initiated from the primers, and once the polymerase reaches the probe, its 5′-3′-exonuclease degrades the probe, physically separating the fluorescent reporter from the quencher, resulting in an increase in fluorescence.

Fluorescence is detected and measured in a real-time PCR machine, and its geometric increase corresponding to exponential increase of the product is used to determine the quantification cycle (Cq) in each reaction.

Real-time RT-PCR assays for SARS-CoV-2 RNA detection were exemplified below using QuantiNova Probe RT-PCR Kit (Qiagen), and be used in a LightCycler 480 Real-Time PCR System (Roche, Basel, Switzerland) Each reaction mixture contained QuantiNova Probe RT-PCR Master Mix, QN Probe RT-Mix, forward and reverse primer, probe, 4 μl TNA as the template. Thermal cycling is exemplified at 45° C. for 10 min for reverse transcription, followed by 95° C. for 5 min and then 45 cycles of 95° C. for 5 s, 55° C. for 30 s.

In preferred embodiments, the disclosed methods are sensitive and/or specific for detection of SARS-CoV-2 in a sample. Preferable, positive detection of SAR-CoV-2 is accompanied by the absence of detection of (i.e., negative for), other human- and/or non-human pathogenic coronaviruses or respiratory pathogens, including, but not limited to, (SARS-CoV, MERS-CoV, HCoV-229E, HCoV-NL63, HCoV-OC43), and 12 virus culture isolates of other respiratory viruses (Influenza virus A[H1N1] and A[H3N2], influenza B virus, influenza C virus, rhinovirus, adenovirus, respiratory syncytial virus, human metapneumovirus and parainfluenza virus types 1-4. In preferred embodiments, the methods using RT-PCR and SEQ ID Nos. 1-3) are more sensitive for SARS-CoV-2 the methods RT-PCR and SEQ ID Nos. 4, 5 and 6) (Table 2).

Exemplary assays were carried out as discussed in the examples below, using the primers and probes provided in Table 1.

In some embodiments, the assays are multiplexed to detect two or more target sequences (e.g., RdRp/helicase (Hel), Spike (S), E, ORF1a/b and/or Nucleocapsid (N)), in addition to NSP1, at once. SARS-CoV-2 has four structural proteins, known as the S (spike), E (envelope), M (membrane), and N (nucleocapsid) proteins; the N protein holds the RNA genome, and the S, E, and M proteins together create the viral envelope. The incorporation of the NSP1 gene in multiplex RT-PCR assays can be used to improve the detection of SARS-CoV-2 using these other targets. NSP1 is a novel 5′ end gene target for molecular detection of SARS-CoV-2. The addition of NSP1 for multiplex detection of SARS-CoV-2 can avoid false negative results due to mutations at the primers/probes binding sites of currently available RT-PCR assays.

As demonstrated in the Examples below using a total of 101 archived respiratory tract specimens (which tested positive for SARS-CoV-2 a RdRp/Hel assay), SARS-CoV-2 was detected by at least one of nsp1, N or E gene RT-PCR in 99 patients (98.0%), and 85 patients (84.2%) were detected by all 3 RT-PCR assays (Table 3). Two patients (2%) were positive by nsp1 gene RT-PCR only. The sensitivity was 93.1% for nsp1 gene RT-PCR, 95.1% for N gene RT-PCR, and 89.1% for E gene RT-PCR, while the specificity was 100% for all 3 RT-PCR assays (Table 4). Accordingly, the inclusion of the NSP1 gene in a multiplex assay would reduce incidences of false negative results.

C. Diagnostic Methods

Diagnostic methods are also provided and can include subjecting a biological sample obtained from the subject (or e.g., total nucleic acid or RNA prepared therefrom) to a SARS-CoV-2 detection method described herein and diagnosing the subject as having SARS-CoV-2 if SARS-CoV-2 (e.g., the target gene such as SARS-CoV-2 RdRp/helicase (Hel), Spike (S), and/or Nucleocapsid (N)) is/are detected.

### IV. Kits

1. Kits for use with the methods disclosed herein are also disclosed. The kits typically include one or more reagents for lysing cells, isolating nucleic acids, particularly RNA, from cell lysate, reverse transcription, second strand synthesis, purifying cDNA, PCR, or any combination thereof.

Reagents can be, for example, buffers, primers, probes, enzymes, dNTPs, carrier RNA, and other active agents and organics that facilitate various steps of the disclosed reactions. The kits can also include instructions for use.

### Examples

Methods

### Clinical Specimens

For nanopore sequencing, t10 original clinical specimens of 9 patients with high viral load were used. For the determination of analytical specificity, the total nucleic acid (TNA) that were extracted from 13 nasopharyngeal aspirate specimens previously found to be positive for human coronaviruses (OC43, NL63, HKU1, 229E) by FilmArray® Respiratory Panel 2 (RP2) (BioFire®, Biomerieux) was used. These specimens were collected between November 2018 and December 2019.

TABLE 1
Nasopharyngeal aspirate specimens positive for coronaviruses that
were used in the analytical specificity of the nsp1 RT-PCR.

| | FilmArray RP2 result | | | |
|---|---|---|---|---|
| | | Other | Sex/ | |
| Specimen | | respiratory | Age in | Date of |
| number | Coronavirus | viruses | years | collection |
| FARR_055 | OC43 | Parainfluenza | F/63 | 2018 Nov. 8 |
| | | virus type 4 | | |
| FARR_059 | NL63 | Parainfluenza | M/56 | 2018 Nov. 29 |
| | | virus type 4 | | |
| FARR_060 | NL63 | Nil | M/84 | 2018 Aug. 27 |
| FARR_067 | HKU1 | Nil | M/61 | 2018 Dec. 31 |
| FARR_155 | 229E | Respiratory | F/13 | 2019 Mar. 24 |
| | | syncytial | | |
| | | virus | | |
| FARR_186 | HKU1 | Parainfluenza | M/1 | 2019 Apr. 20 |
| | | virus type 3 | | |
| FARR_217 | HKU1 | Nil | M/1 | 2019 May 6 |
| FARR_247 | HKU1 | Parainfluenza | F/46 | 2019 Jun. 10 |
| | | virus type 3; | | |
| | | rhinovirus/ | | |
| | | enterovirus | | |
| FARR_314 | NL63 | Nil | M/59 | 2019 Aug. 13 |
| FARR_322 | 229E | Respiratory | M/47 | 2019 Sep. 4 |
| | | syncytial | | |
| | | virus | | |
| FARR_477 | HKU1 | Nil | M/72 | 2019 Dec. 20 |
| FARR_481 | OC43 | Nil | M/80 | 2019 Dec. 24 |
| FARR_494 | 229E | Nil | F/50 | 2019 Dec. 31 |

For clinical validation, we retrieved specimens that tested positive for SARS-CoV-2 using our-house RdRp/Hel assay reported previously [14]. The archived TNA was extracted from clinical specimens collected from COVID-19 patients admitted to Queen Mary Hospital, or from COVID-19 patients admitted to Princess Margaret Hospital for whom specimens were sent to Queen Mary Hospital for viral load testing. For the 88 patients of Queen Mary Hospital, the specimens were collected within 24 hours after hospital admission. For the 14 patients from Princess Margaret Hospital, we have retrieved their first specimens that were sent to our laboratory for viral load testing. We have also retrieved nasopharyngeal specimens that tested negative by LightMix® Modular SARS and CoV E-gene kit. The TNA of these specimens were extracted using NucliSENS easyMAG extraction system (BioMerieux, Marcy l'Étoile, France). Specimens with insufficient volume of total nucleic acid were excluded from analysis.

This study was approved by the Institutional Review Board of the University of Hong Kong/Hospital Authority Hong Kong West Cluster (HKU/HK HKW IRB) (UW 13-372). This study is reported according to Standards for Reporting of Diagnostic Accuracy Studies (STARD) guideline [20].

### Nanopore Sequencing Library

Nanopore sequencing was performed as we described previously with modifications [2,3]. To deplete host cells, nasopharyngeal or saliva specimens were centrifuged at 16,000×g for 2 min, and supernatant was used for subsequent RNA extraction. RNA was extracted from 140 μL of supernatant using QIAamp Viral RNA Mini Kit (Qiagen, Hilden, Germany) as we described previously. RNA was DNase treated, concentrated and cleaned using RNA Clean & Concentrator-5 (Zymo Research, Irvine, Calif.).

Sequence-independent single-primer amplification (SISPA) was performed as described previously [2]. Briefly, DNase-treated RNA was reverse transcribed to single strand cDNA using primer A (5′-GTTTCCCACTGGAGGATA-N9-3′)(SEQ ID NO:8). Second strand cDNA synthesis was performed using Klenow Fragment (3′→5′ exo-) (New England BioLabs, Ipswich, Mass.). PCR using primer B (5′-GTTTCCCACTGGAGGATA-3′) (SEQ ID NO:9) was used in generating the amplified cDNA libraries. Nanopore sequencing library preparation was performed according to manufacturer's instructions for Ligation Sequencing Kit (SQK-LSK109, Oxford Nanopore Technologies). Briefly, amplified PCR products were purified by 1×AM-Pure XP bead (Beckman Coulter, California, CA). Equal molar of each amplified PCR products were then subjected to DNA repair, end preparation, and native barcode ligation (EXP-NBD104, Oxford Nanopore Technologies). Barcoded samples were pooled and were ligated to sequencing adaptor. Sequencing was performed with Oxford Nanopore MinION device using R9.4.1 flow cell for 12-48 hours.

After sequencing, Guppy v3.4.5 was used in converting the raw signal data into FASTQ format, demultiplexing, removal of nanopore and SISPA adaptor sequences. Only reads with a minimum Q score of 7 were included for subsequent analysis. The sequencing run was quality-checked using MinIONQC [21]. Human reads were depleted by mapping to reference human genome hg38, and unmapped reads were extracted using SAMTools [22]. The non-human reads were mapped to the reference genome SARS-CoV-2 isolate (NCBI GenBank: MN908947.3). BCFtools Mpileup was used in creating a variant file [23]. BCFtools call [23], vcfutils.pl [22], and Seqtk seq [24] were used in generating the FASTA consensus sequence. Finally, the coverage data was obtained using SAMtools [22]. Only specimens with a mean coverage of 250× were included for further analysis. Raw reads, after excluding human reads, have been deposited into BioProject.

### Phylogenetic Analysis

The phylogenetic tree of the whole SARS-CoV-2 genome was constructed using neighbor-joining method using MEGA software package version 7.0. The bootstrap values from 1000 replicates were calculated to evaluate the reliability of the phylogenetic trees. Variant A, B and C were defined as described previously [18]. Nucleotide sequences were downloaded from NCBI Genbank and GISAID. The list is detailed below, and more details are available via www.gisaid.org.

| Accession ID | Virus name |
|---|---|
| EPI_ISL_402119 | BetaCoV/Wuhan/IVDC-HB-01/2019 |
| EPI_ISL_402123 | BetaCoV/Wuhan/IPBCAMS-WH-01/2019 |
| EPI_ISL_402129 | BetaCoV/Wuhan/WIV06/2019 |
| EPI_ISL_403932 | BetaCoV/Guangdong/20SF012/2020 |
| EPI_ISL_403933 | BetaCoV/Guangdong/20SF013/2020 |
| EPI_ISL_403935 | BetaCoV/Guangdong/20SF025/2020 |
| EPI_ISL_403936 | BetaCoV/Guangdong/20SF028/2020 |
| EPI_ISL_404228 | BetaCoV/Zhejiang/WZ-02/2020 |
| EPI_ISL_405839 | BetaCoV/Shenzhen/HKU-SZ-005/2020 |
| EPI_ISL_406030 | BetaCoV/Shenzhen/HKU-SZ-002/2020 |
| EPI_ISL_406036 | BetaCoV/USA/CA2/2020 |
| EPI_ISL_406223 | BetaCoV/USA/AZ1/2020 |
| EPI_ISL_406536 | BetaCoV/Foshan/20SF211/2020 |
| EPI_ISL_406593 | BetaCoV/Shenzhen/SZTH-002/2020 |
| EPI_ISL_406597 | BetaCoV/France/IDF0373/2020 |
| EPI_ISL_406596 | BetaCoV/France/IDF0372/2020 |
| EPI_ISL_406862 | BetaCoV/Germany/BavPat1/2020 |
| EPI_ISL_406844 | BetaCoV/Australia/VIC01/2020 |
| EPI_ISL_406798 | BetaCov/Wuhan/WH01/2019 |
| EPI_ISL_406801 | BetaCov/Wuhan/WH04/2020 |
| EPI_ISL_407976 | BetaCoV/Belgium/GHB-03021/2020 |
| EPI_ISL_408480 | BetaCoV/Yunnan/IVDC-YN-003/2020 |
| EPI_ISL_408482 | BetaCoV/Shandong/IVDC-SD-001/2020 |
| EPI_ISL_408484 | BetaCoV/Sichuan/IVDC-SC-001/2020 |
| EPI_ISL_408488 | BetaCoV/Jiangsu/IVDC-JS-001/2020 |
| EPI_ISL_408665 | BetaCoV/Japan/TY-WK-012/2020 |
| EPI_ISL_408666 | BetaCoV/Japan/TY-WK-501/2020 |
| EPI_ISL_408667 | BetaCoV/Japan/TY-WK-521/2020 |
| EPI_ISL_406031 | BetaCoV/Taiwan/2/2020 |
| EPI_ISL_408977 | BetaCoV/Sydney/3/2020 |
| EPI_ISL_409067 | BetaCoV/USA/MA1/2020 |
| EPI_ISL_408478 | BetaCoV/Chongqing/YC01/2020 |
| EPI_ISL_410218 | BetaCov/Taiwan/NTU02/2020 |
| EPI_ISL_410532 | BetaCoV/Japan/OS-20-07-1/2020 |
| EPI_ISL_410536 | BetaCoV/Singapore/5/2020 |
| EPI_ISL_407215 | BetaCoV/USA/WA1-F6/2020 |
| EPI_ISL_410717 | BetaCoV/Australia/QLD03/2020 |
| EPI_ISL_410713 | BetaCoV/Singapore/7/2020 |
| EPI_ISL_410714 | BetaCoV/Singapore/8/2020 |
| EPI_ISL_410545 | BetaCoV/Italy/INMI1-isl/2020 |
| EPI_ISL_410546 | BetaCoV/Italy/INMI1-cs/2020 |
| EPI_ISL_410720 | BetaCoV/France/IDF0372-isl/2020 |
| EPI_ISL_411060 | BetaCoV/Fujian/8/2020 |
| EPI_ISL_411219 | BetaCoV/France/IDF0386-islP1/2020 |
| EPI_ISL_411927 | BetaCoV/Taiwan/4/2020 |
| EPI_ISL_411929 | BetaCoV/South Korea/SNU01/2020 |
| EPI_ISL_411956 | BetaCoV/USA/TX1/2020 |
| EPI_ISL_411951 | BetaCoV/Sweden/01/2020 |
| EPI_ISL_412026 | BetaCoV/Hefei/2/2020 |
| EPI_ISL_412029 | BetaCoV/Hong Kong/VM20001988/2020 |
| EPI_ISL_412030 | BetaCoV/Hong Kong/VB20026565/2020 |
| EPI_ISL_412116 | BetaCoV/England/09c/2020 |
| EPI_ISL_412872 | BetaCoV/Korea/KCDC12/2020 |
| EPI_ISL_412978 | hCoV-19/Wuhan/HBCDC-HB-02/2020 |
| EPI_ISL_413014 | hCoV-19/Canada/ON-PHL2445/2020 |

### Selection of Primers and Probes

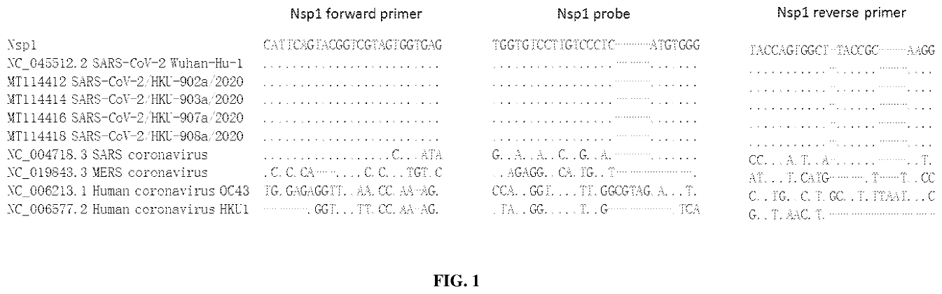

Primers and probes targeting the nsp1 region was designed by multiple alignment of SARS-CoV-2 and other human coronaviruses in the genus Betacoronavirus, including lineage A HCoV-OC43 and HCoV-HKU1, lineage B SARS-CoV, and lineage C MERS-CoV (FIG. 1).

### Analytical Sensitivity and Specificity

The limit of detection (LOD) was determined using serially-diluted SARS-CoV-2 virus culture isolates as described previously [14,25]. SARS-CoV-2 virus was cultured in VeroE6 cells. The concentration of the virus culture stock was 1.8×10⁷ 50% tissue culture infective doses (TCID50)/mL. Triplicates were performed for each dilution in 2 independent experiments.

Analytical specificity was determined using 13 clinical specimens positive for human coronaviruses 229E (n=3), NL63 (n=3), OC43 (n=2) and HKU1 (n=5), and from 17 virus culture isolates of SARS-CoV, MERS-CoV, HCoV-229E, HCoV-NL63, HCoV-OC43, influenza viruses (A[H1N1], A[H3N2], B, C), respiratory syncytial virus, parainfluenza viruses 1-4, human metapneumovirus, rhinovirus/enterovirus and adenovirus as described previously [25].

### Real-Time RT-PCR for Nsp1 Gene

Real time RT-PCR was performed by QuantiNova Probe RT-PCR Kit (Qiagen, Hilden, Germany). A 20 μl reaction containing 4 μl of TNA, 10 μl 2× QuantiNova Probe RT-PCR Master Mix, 0.2 μl QN Probe RT Mix, 1.6 μl of each 10 μM forward and reverse primer, 0.4 μl of 10 μM probe, and 2.2 μl nuclease-free water. Thermal cycling was performed at 45° C. for 10 min for reverse transcription, followed by 95° C. for 5 min and then 45 cycles of 95° C. for 5 s, 55° C. for 30 s. The sequences of primers and probes are listed in Table 2.

TABLE 2
Primers and probes used in this study

| Target (Source) | Primer/Probe | Sequence (5’-3’) |
|---|---|---|
| nsp1 gene (This study) | Forward | CATTCAGTACGGTCGTAGTGGTGAG |
| | | (SEQ ID NO: 1) |
| nsp1 gene (This study) | Reverse | CCTTGCGGTAAGCCACTGGTA |
| | | (SEQ ID NO: 2) |
| nsp1 gene (This study) | Probe | FAM- |
| | | CCCACATGAGGGACAAGGACACCA- |
| | | IABkFQ (SEQ ID NO: 7) |
| E gene [Corman et al] | Forward | ACAGGTACGTTAATAGTTAATAGCGT |
| | | (SEQ ID NO: 4) |
| E gene [Corman et al] | Reverse | ATATTGCAGCAGTACGCACACA |
| | | (SEQ ID NO: 5) |
| E gene [Corman et al] | Probe | FAM- |
| | | ACACTAGCCATCCTTACTGCGCTTCG- |
| | | BBQ (SEQ ID NO: 6) |

Abbreviations: nsp1, non-structural protein 1; E, envelope

### Real-Time RT-PCR for E Gene

Real-time RT-PCR for E gene was performed as described previously, except that the total reaction volume was reduced to 20 μl instead of 25 μl [13,26]. Briefly, superscript III one-step RT-PCR system with Platinum™ Taq Polymerase (Thermo Fisher Scientific, Waltham, Mass., USA) was used. A 20 μl reaction containing 4 μl of TNA, 10 μl 2× Reaction mix (containing 0.4 mM of each deoxyribonucleotide triphosphates (dNTP) and 3.2 mM magnesium sulfate), 0.8 μg of nonacetylated bovine albumin, 0.32 μl of a 50 mM magnesium sulfate solution, 0.8 μl of each 10 μM forward and reverse primer, 0.4 μl of 10 μM probe and 0.8 μl of Superscript III reverse transcriptase/Platinum Taq Mix. Thermal cycling was performed at 55° C. for 10 min for reverse transcription, followed by 95° C. for 3 min and then 45 cycles of 95° C. for 15 s, 58° C. for 30 s.

### Statistical Analysis

Statistical analysis was performed using PRISM® 6.0 for Windows. The sensitivity of nsp1 and E gene real-time RT-PCT was compared using Fisher's exact test.

### Results

### Nanopore Sequencing of SISPA-Amplified Genome

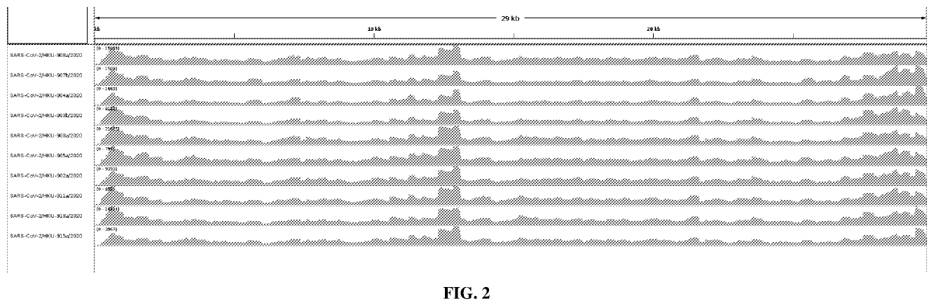

Nanopore sequencing of SISPA-amplified genome was performed for a total of 22 specimens from 14 patients. Ten specimens from 9 patients had a coverage of 250× and were included for further analysis. The consensus sequences of four patients were previously deposited into NCBI GenBank (AMT114412, MT114414-MT114415, MT114417-MT114418) [3]. For one patient, both the nasopharyngeal and saliva specimen was included. For the other 8 patients, there were 3 saliva and 5 nasopharyngeal specimens. All patients were hospitalized at Princess Margaret Hospital. The median age was 62 years. Four patients were female. Four patients required oxygen supplementation, 2 patients were admitted to the intensive care unit, and 1 patient died. Phylogenetic analysis using the whole viral genome showed that the virus strains from 2 patients belonged to variant A, and those from 7 patients belonged to variant B (data not shown). To identify gene regions that were highly expressed, coverage map was visualized using integrative genomics viewer (IGV). The expression of different gene regions was similar among these 9 patients (FIG. 2) (nucleotide positions indicated in Supplementary Table 3)

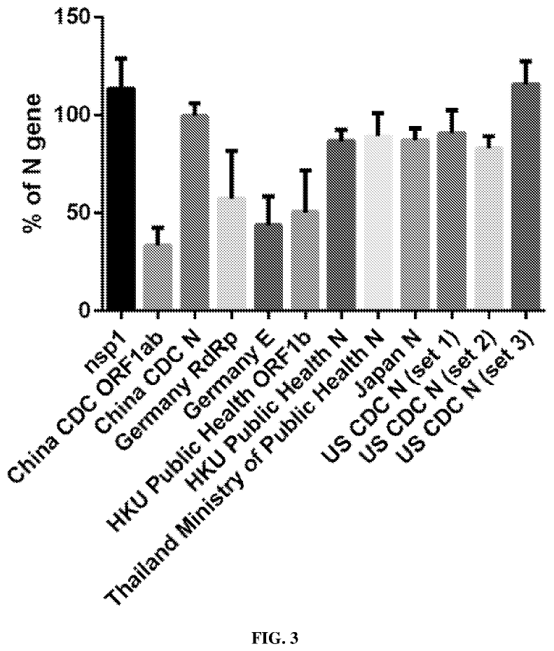

TABLE 3
Original source of the real-time reverse transcription polymerase
chain reaction assay used in FIG. 3 (https://www.who.int/docs/default-
source/coronaviruse/whoinhouseassays.pdf?sfvrsn=de3a76aa_2).

| Organization | Target gene | Nucleotide position^{a} |
|---|---|---|
| China CDC, China | ORF1ab(nsp10/11 ) | 13342-13460 |
| | N | 28881-28979 |
| Charité, Germany | RdRp | 15431-15530 |
| | E | 26269-26381 |
| HKU, Hong Kong SAR | ORF1B (nsp14) | 18778-18909 |
| | N | 29145-29254 |
| National Institute of | N | 28320-28376 |
| Health, Thailand | | |
| National Institute of | N | 29125-29282 |
| Infectious Diseases, | | |
| Japan | | |
| US CDC, USA | N (Set 1) | 28287-28358 |
| | N (Set 2) | 29164-29230 |
| | N (Set 3) | 28681-28752 |

* The nucleotide numbering are based on reference SARS-CoV-2 genome isolate (NCBI Reference Sequence: NC_045512.2)

### Nsp1 Primers and Probes

Specific primers and probes targeting SARS-CoV-2 were selected by aligning SARS-CoV-2 sequences and other human betacoronaviruses (FIG. 1). Analysis showed that our nsp1 real-time RT-PCR target region was expressed more abundantly than the gene targets of other published real time RT-PCR assay (FIG. 3).

### Analytical Sensitivity and Specificity

The LOD of nsp1 was 18 TCID₅₀/ml. Nsp1 RT-PCR had tested negative for all 13 clinical specimens known to be positive for coronaviruses, 5 virus culture isolates of coronavirus (SARS-CoV, MERS-CoV, HCoV-229E, HCoV-NL63, HCoV-OC43), and 12 virus culture isolates of other respiratory viruses (Influenza virus A[H1N1] and A[H3N2], influenza B virus, influenza C virus, rhinovirus, adenovirus, respiratory syncytial virus, human metapneumovirus and parainfluenza virus types 1-4).

### Diagnostic Performance of Nsp1 RT-PCR

A total of 101 archived respiratory tract specimens collected between 29 Feb. and 7 Apr. 2020, and tested positive for SARS-CoV-2 by an RdRp/Hel assay, were retrieved. The specimens included 36 nasopharyngeal aspirate/swab, 35 combined nasopharyngeal and throat swab, 27 posterior oropharyngeal saliva, 2 throat swab, and 1 endotracheal aspirate. Of these 101 COVID-19 patients, SARS-CoV-2 was detected by at least one of nsp1, N or E gene RT-PCR in 99 patients (98.0%), and 85 patients (84.2%) were detected by all 3 RT-PCR assays (Table 4).

TABLE 4
Concordance of nsp1, N and E gene RT-PCR assays.

| RT-PCR | | | Number (%) | |
|---|---|---|---|---|
| nsp1 | N | E | (n = 101) | |
| + | + | + | 85 | (84.2) |
| + | + | − | 6 | (5.9) |
| + | − | + | 1 | (1.0) |
| + | − | − | 2 | (2.0) |
| − | + | + | 4 | (4.0) |
| − | + | − | 1 | (1.0) |
| − | − | + | 0 | (0) |
| − | − | − | 2 | (2.0) |

Abbreviations:
+, positive;
−, negative

Two patients (2%) were positive by nsp1 gene RT-PCR only. The sensitivity was 93.1% for nsp1 gene RT-PCR, 95.1% for N gene RT-PCR, and 89.1% for E gene RT-PCR, while the specificity was 100% for all 3 RT-PCR assays (Table 5).

TABLE 5
Sensitivity and specificity of nsp1 gene RT-PCR when
compared with those of E gene and N gene RT-PCR.

| | Patients | Patients | | |
|---|---|---|---|---|
| | with | without | Sensitivity | Specificity |
| RT-PCR | COVID-19 | COVID-19 | (95% CI) | (95% CI) |
| target | (n = 101)* | (n = 50) | (%) | (%) |
| nsp1 | 94 | 0 | 93.1 | 100 |
| | | | (86.2-97.2) | (92.9-100) |
| N | 96 | 0 | 95.1 | 100 |
| | | | (88.8-98.4) | (92.9-100) |
| E | 90 | 0 | 89.1 | 100 |
| | | | (81.3-94.4) | (92.9-100) |

*Detected by RdRp-Hel RT-PCR
Abbreviations: CI, confidence interval

### Discussion

In this study, a highly sensitive and specific nsp1 real-time RT-PCR for the detection of SARS-CoV-2 was developed. The studies first identified nsp1 to be a highly expressed gene target in clinical specimens using nanopore whole genome sequencing, and designed a real-time RT-PCR protocol based on nsp1 gene. This novel nsp1 real-time RT-PCR has a low limit of detection, and did not cross react with other human coronaviruses or other respiratory viruses. The nsp1 real-time RT-PCR has a sensitivity of 93.1%. The nsp1 RT-PCR was also highly specific.

Identifying alternative targets for the detection of SARS-CoV-2 is important because genetic variations can affect the sensitivity of RT-PCR. There are two major sources of genetic variations in coronaviruses. First, due to the lack of proofreading function, RNA polymerase are prone to error, and single nucleotide polymorphisms occur frequently. It was estimated that the average substitution rate of SARS-CoV and MERS-CoV is about 10⁻³ substitutions per year per site [27]. Second, recombination is well known to occur for coronaviruses. These recombination events can occur between viruses in the same genus, same lineage, or even in different lineages [27]. The recombination occurs in both animal and human coronaviruses [28,29]. SARS-CoV-2 is also believed to arise due to recombination event that involves the receptor binding motif of a closely related BatcoV RaTG13 [30]. Recombination is especially important because the current real-time RT-PCR targets are mainly located in the middle or 3′ end of the genome. In contrast, nsp1 gene is located in the 5′ end, and therefore may not be affected by recombination events occurring between the nsp1 gene and RdRp gene. Unlike other targets commonly used for diagnosis, the nsp1 is located in the 5′ end of the genome. These gene targets are located in the middle or 3′ end of the viral genome [31]. Since recombination can occur, it is important to have a target at the 5′ end of the genome.

Nsp1 is usually not considered to be highly expressed. However, our nanopore sequencing of clinical specimens have consistently shown that this gene region is highly expressed. Traditionally, it was thought that the subgenomic sequences arise from the leader sequence. However, recently, it was shown that the subgenomic sequences arise at the nsp1-truncated nsp2 and/or truncated nsp3 region (DOI: 10.1016/j.cell.2020.04.011). We have also demonstrated that nsp2 showed 100% concordance with RdRp/Hel RT-PCR [25].

Shortly after the announcement of SARS-CoV-2 as the causative pathogen of COVID-19, Shirato et al has reported the use of a conventional nested RT-PCR targeting nsp1 gene, but require Sanger sequencing for confirmation [32]. In this early report, the sensitivity and specificity of their conventional nsp1 gene nested RT-PCR was not reported.

The coverage map for SARS-CoV-2 from our study using of SISPA and nanopore sequencing suggests that nsp1 was highly expressed in clinical specimens. In contrast, in a previous study, nsp1 was not shown to be highly expressed in the coverage map from Illumina sequencing [33]. One possibility is the difference in library preparation. In the paper by Wu et al, ribosomal RNA depletion was performed to reduce the amount of human RNA in the specimen before reverse transcription. Ribosomal RNA depletion has been shown to introduce biased distribution of read coverage for influenza virus [34]. In contrast, our library preparation did not involve ribosomal RNA depletion, which may have avoided this bias.

Another potential technique to survey expression is direct RNA sequencing using nanopore technology. However, this technique requires a large amount of high quality and pure viral RNA as starting material, which is often unfeasible from clinical specimens. Moreover, the protocol utilizes oligodT primers to capture the polyA tail to allow the complex to be brought to the sequencing pore where the RNA is read from the 3′ to 5′ direction. This technology may not be useful in assessing abundance of particular regions as efficiency of detection is higher in early reads resulting in coverage bias towards the 3′ end of the genome.

Studies from SARS-CoV indicate that NSP1 suppresses the antiviral response [31]. NSP1 can downregulate host gene expression by binding to the 40S ribosome to block the assembly of translationally competent ribosome, and then inducing endonucleolytic cleavage and the degradation of host mRNAs; and by altering the nuclear-cytoplasmic distribution of an RNA binding protein, nucleolin [35]. Since nsp1 gene is highly expressed in our SARS-CoV-2 patients, it remains to be determined whether the function of NSP1 is also enhanced in SARS-CoV-2.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

### REFERENCES

[1] Peiris J S M, Lai S T, Poon L L M, Guan Y, Yam L Y C, Lim W, et al. Coronavirus as a possible cause of severe acute respiratory syndrome. Lancet 2003; 361:1319-25
[2] Chan J F, Yuan S, Kok K H, To K K, Chu H, Yang J, et al. A familial cluster of pneumonia associated with the 2019 novel coronavirus indicating person-to-person transmission: a study of a family cluster. Lancet 2020; 10.1016/S0140-6736(20)30154-9
[3] To K K, Tsang O T, Leung W S, Tam A R, Wu T C, Lung D C, et al. Temporal profiles of viral load in posterior oropharyngeal saliva samples and serum antibody responses during infection by SARS-CoV-2: an observational cohort study. Lancet Infect Dis 2020; 10.1016/S1473-3099(20)30196-1
[4] Cheung K S, Hung I F, Chan P P, Lung K C, Tso E, Liu R, et al. Gastrointestinal Manifestations of SARS-CoV-2 Infection and Virus Load in Fecal Samples from the Hong Kong Cohort and Systematic Review and Meta-analysis. Gastroenterology 2020; 10.1053/j.gastro.2020.03.065
[5] Shi H, Han X, Jiang N, Cao Y, Alwalid O, Gu J, et al. Radiological findings from 81 patients with COVID-19 pneumonia in Wuhan, China: a descriptive study. Lancet Infect Dis 2020; 10.1016/S1473-3099(20)30086-4
[6] Huang C, Wang Y, Li X, Ren L, Zhao J, Hu Y, et al. Clinical features of patients infected with 2019 novel coronavirus in Wuhan, China. Lancet 2020; 395:497-506
[7] Wang D, Hu B, Hu C, Zhu F, Liu X, Zhang J, et al. Clinical Characteristics of 138 Hospitalized Patients With 2019 Novel Coronavirus-Infected Pneumonia in Wuhan, China. JAMA 2020; 10.1001/jama.2020.1585
[8] Chen N, Zhou M, Dong X, Qu J, Gong F, Han Y, et al. Epidemiological and clinical characteristics of 99 cases of 2019 novel coronavirus pneumonia in Wuhan, China: a descriptive study. Lancet 2020; 395:507-13
[9] Yang X, Yu Y, Xu J, Shu H, Xia J, Liu H, et al. Clinical course and outcomes of critically ill patients with SARS-CoV-2 pneumonia in Wuhan, China: a single-centered, retrospective, observational study. The lancet Respiratory medicine 2020; 10.1016/S2213-2600(20)30079-5
[10] Xu Z, Shi L, Wang Y, Zhang J, Huang L, Zhang C, et al. Pathological findings of COVID-19 associated with acute respiratory distress syndrome. The lancet Respiratory medicine 2020; 10.1016/S2213-2600(20)30076-X
[11] Sharfstein J M, Becker S J, Mello M M. Diagnostic Testing for the Novel Coronavirus. JAMA 2020; 10.1001/jama.2020.3864
[12] Li R, Pei S, Chen B, Song Y, Zhang T, Yang W, et al. Substantial undocumented infection facilitates the rapid dissemination of novel coronavirus (SARS-CoV2). Science 2020; 10.1126/science.abb3221
[13] World Health organization. Coronavirus disease (COVID-19) technical guidance: Laboratory testing for 2019-nCoV in humans. Available at https://www.who.int/docs/default-source/coronaviruse/whoinhouseassays.pdf?sfvrsn=de3a76aa_2. Accessed on Apr. 18, 2020. 2020
[14] Chan J F, Yip C C, To K K, Tang T H, Wong S C, Leung K H, et al. Improved molecular diagnosis of COVID-19 by the novel, highly sensitive and specific COVID-19-RdRp/Hel real-time reverse transcription-polymerase chain reaction assay validated in vitro and with clinical specimens. J Clin Microbiol 2020; 10.1128/JCM.00310-20
[15] Chan J F, Lau S K, To K K, Cheng V C, Woo P C, Yuen K Y. Middle East respiratory syndrome coronavirus: another zoonotic betacoronavirus causing SARS-like disease. Clin Microbiol Rev 2015; 28:465-522
[16] Cheng V C, Lau S K, Woo P C, Yuen K Y. Severe acute respiratory syndrome coronavirus as an agent of emerging and reemerging infection. Clin Microbiol Rev 2007; 20:660-94
[17] Li X, Wang W, Zhao X, Zai J, Zhao Q, Li Y, et al. Transmission dynamics and evolutionary history of 2019-nCoV. J Med Virol 2020; 92:501-11
[18] Forster P, Forster L, Renfrew C, Forster M. Phylogenetic network analysis of SARS-CoV-2 genomes. Proc Natl Acad Sci USA 2020; 10.1073/pnas.2004999117
[19] Yip C C, Chan W M, Ip J D, Seng C W, Leung K H, Poon R W, et al. Nanopore sequencing reveals novel targets for the diagnosis and surveillance of human and avian influenza A virus. J Clin Microbiol 2020; 10.1128/JCM.02127-19
[20] Bossuyt P M, Reitsma J B, Bruns D E, Gatsonis C A, Glasziou P P, Irwig L, et al. STARD 2015: An Updated List of Essential Items for Reporting Diagnostic Accuracy Studies. Radiology 2015; 277:826-32
[21] Lanfear R, Schalamun M, Kainer D, Wang W, Schwessinger B. MinIONQC: fast and simple quality control for MinION sequencing data. Bioinformatics 2019; 35:523-5
[22] Li H, Handsaker B, Wysoker A, Fennell T, Ruan J, Homer N, et al. The Sequence Alignment/Map format and SAMtools. Bioinformatics 2009; 25:2078-9
[23] Narasimhan V, Danecek P, Scally A, Xue Y, Tyler-Smith C, Durbin R. BCFtools/RoH: a hidden Markov model approach for detecting autozygosity from next-generation sequencing data. Bioinformatics 2016; 32:1749-51
[24] https://github.com/lh3/seqtk
[25] Yip C C, Ho C C, Chan J F, To K K, Chan H S, Wong S C, et al. Development of a Novel, Genome Subtraction-Derived, SARS-CoV-2-Specific COVID-19-nsp2 Real-Time RT-PCR Assay and Its Evaluation Using Clinical Specimens. Int J Mol Sci 2020; 21:2574
[26] Corman V M, Landt O, Kaiser M, Molenkamp R, Meijer A, Chu D K W, et al. Detection of 2019 novel coronavirus (2019-nCoV) by real-time RT-PCR. Euro Surveill 2020; 25
[27] Su S, Wong G, Shi W, Liu J, Lai A C K, Zhou J, et al. Epidemiology, Genetic Recombination, and Pathogenesis of Coronaviruses. Trends Microbiol 2016; 24:490-502
[28] Lau S K P, Luk H K H, Wong A C P, Fan R Y Y, Lam C S F, Li K S M, et al. Identification of a Novel Betacoronavirus (Merbecovirus) in Amur Hedgehogs from China. Viruses 2019; 11
[29] Woo P C, Lau S K, Yip C C, Huang Y, Tsoi H W, Chan K H, et al. Comparative analysis of 22 coronavirus HKU1 genomes reveals a novel genotype and evidence of natural recombination in coronavirus HKU1. J Virol 2006; 80:7136-45
[30] Cagliani R, Forni D, Clerici M, Sironi M. Computational inference of selection underlying the evolution of the novel coronavirus, SARS-CoV-2. J Virol 2020; 10.1128/JVI.00411-20
[31] Chan J F, Kok K H, Zhu Z, Chu H, To K K, Yuan S, et al. Genomic characterization of the 2019 novel human-pathogenic coronavirus isolated from a patient with atypical pneumonia after visiting Wuhan. Emerg Microbes Infect 2020; 9:221-36
[32] Shirato K, Nao N, Katano H, Takayama I, Saito S, Kato F, et al. Development of Genetic Diagnostic Methods for Novel Coronavirus 2019 (nCoV-2019) in Japan. Jpn J Infect Dis 2020; 10.7883/yoken.JJID.2020.061
[33] Wu F, Zhao S, Yu B, Chen Y M, Wang W, Song Z G, et al. A new coronavirus associated with human respiratory disease in China. Nature 2020; 579:265-9
[34] Li D, Li Z, Zhou Z, Li Z, Qu X, Xu P, et al. Direct next-generation sequencing of virus-human mixed samples without pretreatment is favorable to recover virus genome. Biol Direct 2016; 11:3
[35] Gomez G N, Abrar F, Dodhia M P, Gonzalez F G, Nag A. SARS coronavirus protein nsp1 disrupts localization of Nup93 from the nuclear pore complex. Biochem Cell Biol 2019; 97:758-66

1. A composition comprising a nucleic acid probe or primer for the detection of SARS-CoV-2 NSP1 gene selected from the group consisting of
(SEQ ID NO: 1)
CATTCAGTACGGTCGTAGTGGTGAG,
(SEQ ID NO: 2)
CCTTGCGGTAAGCCACTGGTA,
(SEQ ID NO: 3)
CCCACATGAGGGACAAGGACACCA,
the reverse complement of any of SEQ ID NOS:1-3, or
a nucleic acid sequence having at least 70%, 75%, 80%, 85%, 90%, 95%, 96%, 97%, 98%, or 99% sequence identity to any of the foregoing.

2. The composition of claim 1, comprising or consisting of the nucleic acid sequence of SEQ ID NO:1, SEQ ID NO:2 or
a nucleic acid sequence having at least 70%, 75%, 80%, 85%, 90%, 95%, 96%, 97%, 98%, or 99% sequence identity to any of the foregoing.

3. The composition of claim 1, comprising or consisting of the nucleic acid sequence of SEQ ID NO:1, SEQ ID NO:2, SEQ ID NO:3 or
a nucleic acid sequence having 1, 2, 3, 4, 5, 6, 7, 8, 9, or 10 nucleic acid substitution(s), addition(s), deletion(s), or a combination thereof relative thereto.

4. The composition of claim 1, comprising a primer pair comprising
a forward primer comprising or consisting of the nucleic acid sequence of SEQ ID NO:1 or a nucleic acid sequence having at least 70%, 75%, 80%, 85%, 90%, 95%, 96%, 97%, 98%, or 99% sequence identity, and
a reverse primer comprising or consisting of the nucleic acid sequence of SEQ ID NO:2 or a nucleic acid sequence having at least 70%, 75%, 80%, 85%, 90%, 95%, 96%, 97%, 98%, or 99% sequence identity, or a nucleic acid sequence having 1, 2, 3, 4, 5, 6, 7, 8, 9, or 10 nucleic acid substitution(s), addition(s), deletion(s), or a combination thereof relative thereto.

5. (canceled)

6. A nucleic acid probe comprising or consisting of the nucleic acid sequence of SEQ ID NO:3 or a nucleic acid sequence having at least 70%, 75%, 80%, 85%, 90%, 95%, 96%, 97%, 98%, or 99% sequence identity, or a nucleic acid sequence having 1, 2, 3, 4, 5, 6, 7, 8, 9, or 10 nucleic acid substitution(s), addition(s), deletion(s), or a combination thereof relative thereto.

7. (canceled)

8. The nucleic acid probe of claim 1 further comprising one or more fluorescent reporters, one or more quenchers, or a combination thereof, optionally comprising a 5′ fluorescent reporter and a 3′ quencher.

9. (canceled)

10. (canceled)

11. (canceled)

12. (canceled)

13. A method of detecting SARS-CoV-2 comprising contacting the sample with the composition of claim 1, optionally wherein the sample is selected from the group consisting of mucus, sputum (processed or unprocessed), bronchial alveolar lavage (BAL), bronchial wash (BW), bodily fluids, cerebrospinal fluid (CSF), urine, tissue (e.g., biopsy material), nasopharyngeal aspirate, nasopharyngeal swab, throat swab, feces, plasma, serum, or whole blood, optionally wherein the sample is processed to isolate nucleic acids.

14. The method of claim 13, wherein the SARS-CoV-2 has a genome comprising the sequence according to GenBank accession no. MN975262 or MN908947.3, or a variant thereof comprising at least 70%, 75%, 80%, 85%, 90%, 95%, 96%, 97%, 98%, or 99% sequence identity thereto.

15. The method of claim 14, wherein the SARS-CoV-2 has a genome comprising or consisting of the sequence according to GenBank accession no. MN975262 or MN908947.3.

16. The method of claim 13, wherein the method of detection comprises analysis by microarray, differential display, RNase protection assay, northern blot, RT-PCR, or a combination thereof.

17. The method of claim 16, further comprising target sequence-specific quantitative or realtime RT-PCR.

18. (canceled)

19. (canceled)

20. The method of claim 15, comprising using nucleic acids of a sample as a template for RT-PCR utilizing the primer pair of comprising SEQ ID NO:1 and SEQ ID NO:2 optionally in combination with a probe comprising SEQ ID NO:3.

21. The method of claim 20, wherein the sample is a biological sample.

22. The method of claim 21, wherein the biological sample is selected from mucus, sputum (processed or unprocessed), bronchial alveolar lavage (BAL), bronchial wash (BW), bodily fluids, cerebrospinal fluid (CSF), urine, tissue (e.g., biopsy material), rectal swab, nasopharyngeal aspirate, nasopharyngeal swab, throat swab, feces, plasma, serum, or whole blood.

23. The method of claim 22, wherein the sample is processed to expose or isolate the nucleic acids.

24. The method of claim 20, wherein the sample is isolated from a subject suspected of having SARS-CoV-2.

25. The method of claim 15 wherein the method is more sensitive, selective, or combination thereof for SARS-CoV-2 relative to one or more other human- and/or non-human pathogenic coronaviruses and/or respiratory pathogens, such as (SARS-CoV, MERS-CoV, HCoV-229E, HCoV-NL63, HCoV-OC43), and 12 virus culture isolates of other respiratory viruses (Influenza virus A[H1N1] and A[H3N2], influenza B virus, influenza C virus, rhinovirus, adenovirus, respiratory syncytial virus, human metapneumovirus and parainfluenza virus types 1-4, and combinations thereof.

26. (canceled)

27. (canceled)


SEQUENCE LISTING (9 sequences)

SEQ ID NO: 1 - DNA, 25 bp, Artificial Sequence
  Forward primer for nsp1 gene of SARS-CoV-2
cattcagtacggtcgtagtggtgag

SEQ ID NO: 2 - DNA, 21 bp, Artificial Sequence
  Reverse primer for nsp1 gene of SARS-CoV-2
ccttgcggtaagccactggta

SEQ ID NO: 3 - DNA, 24 bp, Artificial Sequence
  Probe for nsp1 gene of SARS-CoV-2
cccacatgagggacaaggacacca

SEQ ID NO: 4 - DNA, 26 bp, Artificial Sequence
  Forward primer for E gene of SARS-CoV-2
acaggtacgttaatagttaatagcgt

SEQ ID NO: 5 - DNA, 22 bp, Artificial Sequence
  Reverse primer for E gene of SARS-CoV-2
atattgcagcagtacgcacaca

SEQ ID NO: 6 - DNA, 26 bp, Artificial Sequence
  Probe for E gene of SARS-CoV-2
acactagccatccttactgcgcttcg

SEQ ID NO: 7 - DNA, 24 bp, Artificial Sequence
  Probe for nsp1 gene of SARS-CoV-2
cccacatgagggacaaggacacca

SEQ ID NO: 8 - DNA, 27 bp, Artificial Sequence
  primer A for reverse transcription of RNA
  misc_feature (19)..(27): n is a, c, g, or t
gtttcccactggaggatannnnnnnnn

SEQ ID NO: 9 - DNA, 18 bp, Artificial Sequence
  primer B for generating cDNA libraries
gtttcccactggaggata